(19) United States
(12) Patent Application Publication
(10) Pub. No.: US 2020/0163362 A1
(43) Pub. Date: May 28, 2020

(54) BOLUS FOR ADMINISTRATION TO THE FORESTOMACHS OF RUMINANTS AND METHOD FOR PRODUCING THE BOLUS

(71) Applicant: Ulf-Michael STUMPE, Wriezen (DE)

(72) Inventor: Ulf-Michael STUMPE, Wriezen (DE)

(21) Appl. No.: 16/632,131

(22) PCT Filed: Jul. 19, 2018
(86) PCT No.: PCT/EP2018/069643

§ 371 Date: Jan. 17, 2020

(30) Foreign Application Priority Data
Jul. 20, 2017 (DE) 10 2017 212 520.9

(51) Int. Cl. A23K 10/30; A61K 9/00; A23K 10/18; A23K 20/174; A23K 20/20; A23K 40/35; A23K 50/10

(52) U.S. Cl. CPC A23K 10/30; A61K 9/0068; A23K 10/18; A23K 50/10; A23K 20/30; A23K 40/35; A23K 20/174

(57) ABSTRACT

The invention relates to a bolus for administration to a forestomach of a ruminant, the bolus comprising a mixture of raw fibers and at least one binding agent.

The invention relates to a bolus, which is used for administration to the forestomaches of ruminants. The invention further relates to a method for producing the bolus.

It is known that animals are fed at an energetically very high level for the purposes of fattening and milk production. Thus, the energy density of the feed used is generally significantly higher than that of natural feed, which the animals find in nature. In particular, this applies to beef and dairy cattle, but also to sheep and goats which are kept for milk and meat production. The standards of modern dairy and beef cattle farming are met with the aid of high-energy feed.

In particular, energy for the growth processes and for the production of milk is provided by means of the high-energy feed. However, the use of high-energy feed means that the physiological forestomach functions of ruminants cannot be carried out to an extent that would be necessary with respect to animal health. Therefore, a natural forestomach function in terms of animal welfare is hardly possible. A primary reason for this is that the proportion of raw fiber in the high-performance feed is very low as compared to natural animal feed. An animal-friendly proportion of raw fiber in the feed is necessary for forestomach function in terms of animal welfare, because otherwise acidification of the forestomach system can occur due to free-floating lactic acid bacteria. The effects on animal health are significant: The forestomach flora changes; the appetite, the ruminating activity, and the forestomach activity of the animals reduces sharply; the animal may become sick to the point of it being life-threatening. In addition, there is a negative impact on the immunological capacity due to the acidification.

DE 10 2009 015 558 A1 discloses a bolus for administration into the forestomaches of ruminants, which contains raw fiber. The forestomach peristalsis is supported with the aid of raw fiber, whereby the ruminating activity and the recirculation of the feed components in the forestomach system are improved.

The substances contained in the bolus, particularly the raw fiber, are used to obtain a basis for reestablishing and/or promoting the natural digestive processes, particularly of an animal already ill. Because acidification of the forestomach frequently leads to inflammation, the sick animal avoids taking in structurally-effective raw fibers on its own, for example in the form of hay or grass, because the ends of the raw fibers, for example in the form of stalks, will have a painful effect on the inflamed internal lining of the forestomach. “Structurally-effective” in this context means that the raw fiber has a mechanically activating and stimulating effect on the digestive processes of the forestomach due to its shape, size, and structure. The bolus overcomes the animal's reluctance to take in structurally effective raw fibers and thus reactivate the natural digestive processes necessary for recovery and thus contributes to a relief of illness symptoms and/or to recovery of the sick animal.

Raw fibers, particularly in the form of stalks, stimulate the forestomach in that mechanical stimuli results on the walls of the forestomach. The animals are stimulated to ruminate due to this stimulation. The result of this is that the saliva produced during rumination contributes to the regulation of acidic environmental conditions in the forestomach system and thus counteracts acidification.

DE 10 2009 015 558 B4 proposes a bolus shell to encapsulate the bolus. It is proposed that the bolus shell consists of water-soluble materials, for example in the form of capsules, or even products containing gelatin, cellulose, or protein.

GB 2 353 707 A discloses a bolus for ruminants, which is partially or completely coated with a material that is insoluble or barely soluble in water. In use, the coating is removed from the bolus progressively due to abrasion from the reticulorumen.

A bolus according to the prior art becomes unusable as soon as the shell or coating has a hole or a tear, for example resulting during an unsuccessful attempt to bring food into the mouth of the animal or during the act of swallowing. This also carries the risk that components of the bolus could spread uncontrollably in the mouth or throat area of the animal and thus also could reach the airway. This particularly applies to a bolus the components of which are not connected to one another or only connected loosely, thus in the case of a bolus consisting solely of raw fiber.

The application of a shell or a coating further complicates the production methods for boli. In turn, this particularly applies to those boli containing components that are not connected to one another or only connected loosely. In particular, they may be raw fibers separated in the original state. Molding the components not connected to one another or only connected loosely during production for the purposes of subsequent application of a shell or a coating is difficult.

The object of the invention is to implement a bolus containing raw fiber such that it does not have the aforementioned problems, particularly without a shell or a coating and particularly before and during an act of swallowing by an animal.

A basic concept of the present invention is the mixing of raw fiber with a binding agent such that the components of the raw fiber are connected to one another, basically both on the surface and inside of the bolus.

With a bolus of the invention, it is possible to stabilize and/or stimulate the feed consumption by the animal, which then means that the raw fiber concentration will be available to a physiological extent. If forestomach fermentation is derailed, this results in some negative systemic metabolic situations which, in turn, lead to metabolic reduction of appetite not mechanically but pathophysiologically. This can be counteracted with a bolus according to the invention.

The uncontrolled swelling of raw fiber proportions can be reduced by stabilizing the shape when the air humidity or moisture in the mouth cavity acts on the raw fiber bolus.

In particular, a bolus according to claim 1 is proposed in order to achieve the first object: a bolus for administration into a forestomach of a ruminant, wherein the bolus has a mixture of raw fiber and at least one binding agent, or consists thereof, or substantially consists thereof.

As a proportion of the mixture of raw fiber and the at least one binding agent, the parts of the raw fiber are connected to one another due to the at least one binding agent. On the one hand, this is advantageous because the production method of the bolus, which will be addressed in more detail later, is simplified. Thus, the complicated step of having to apply a coating or a shell to components of the bolus not connected to one another or only loosely connected is omitted. On the other hand, this results in the advantage that external damage, for example a hole or a tear, will not make the bolus unusable, because the shape and the structure of the bolus does not change or not substantially despite the damage. Such type of damage may particularly result when introducing food into the mouth of the animal and during an act of swallowing.

There is a further substantial advantage in this context: The risk that the bolus is destroyed or becomes damaged when introduced into the mouth of the animal, which takes place by means of customary administration aids, or during an act of swallowing is reduced to a minimum. Thus, it is highly improbable that components of the bolus could spread uncontrollably in the mouth or throat area of the animal and thus also could reach the airway. The negative consequences, on the one hand, that components of the bolus valuable to the animal do not reach the forestomach and, on the other hand, that the animal will have difficulty breathing and/or even respiratory distress to the point of choking are extensively prevented.

A further advantage is that the mixture of raw fiber and at least one binding agent means that the bolus can be transported easily through the esophagus of the animal. This is particularly true when at least one binding agent and a binding agent quantity is used that leads, for example, to a smooth or clearly defined and/or well-sliding surface of the bolus, for example fat. Further possible binding agents are mentioned in the following.

The term “raw fiber” in this context may designate natural fibrous substances; this includes particularly plant parts, as will be explained in more detail in the following. The term “raw fiber” may also characterize artificial fibrous substances, for example synthetic fibrous cellulose.

The at least one binding agent may essentially be a structure-providing substance or several structure-providing substances. The term “structure-providing” means that the bolus retains or contains a stable shape due to the binding agent to the point it is destroyed at the desired location in the forestomach of a ruminant.

The binding agent may provide the structural integrity to the composite of raw fiber and binding agent. This means, in particular, that the substance/substances do not form any or only a few defects within the bolus in the form of tears or similar defects under the effect of considerable external pressure, optionally under the effect of a temperature higher than the environment, and that the substance/substances thus adhere together easily and durably.

Structural integrity of the bolus can be achieved, even in unfavorable storage conditions, for example storage with high humidity, extensive dryness, or heat, with the aid of the at least one binding agent. Such type of structural integrity could not be achieved solely through the use of only raw fiber without the at least one binding agent.

The at least one binding agent should not be understood in terms of a shell but rather a substance that is distributed, preferably evenly, and is contained in a predominant portion of the volume, preferably in the entire volume of the bolus. This essentially includes the outer surfaces of the bolus and all internal areas. In other words, the mixture of raw fiber and binding agent extends into the interior of the bolus, preferably over the entire volume of the bolus. Preferably, the bolus has a uniform or substantially uniform mixture of raw fiber and binding agent.

Individual raw fibers or raw fiber components are connected to one another by the binding agent.

Raw fibers or raw fiber components may be completely or partially enclosed by binding agent and/or completely or partially embedded in the binding agent. Raw fibers or raw fiber components may partially protrude from the surface on the outer side of the bolus.

The ratio of raw fiber to binding agent may be selected variably. The ratio is preferably selected such that a bolus with structural integrity is obtained. In particular, the ratio is preferably selected such that there are no raw fibers not connected to other raw fibers through the binding agent.

The bolus may have cavities that are filled with raw fiber or with binding agent.

In addition to the at least one binding agent, the bolus may have a bolus shell. The bolus shell may consist of a substance that decomposes in liquid, for example in the forestomach.

The bolus shell may consist of any animal-friendly substances, particularly of cellulose, proteins, gelatin, or starchy substances. A bolus shell made from a fat-containing metabolic preparation for administration in the event of energy deficiencies, i.e. a supplementary feedstuff, is conceivable, for example Ket 40, which is a brand from ufamed AG. In this form, the bolus shell additionally offers an energetic component.

Furthermore, the bolus and/or the at least one binding agent may have at least one further substance, which is required for animal health and/or has another function. Various examples of this are found in the embodiments of the invention, which are proposed in the following.

Due to the fact that the bolus, particularly the at least one binding agent, has at least one further substance, further states of illness of an animal can be cured or relieved, in addition to the deficiency of structurally effective raw fiber proportions and the consequences thereof. This relates particularly to acidification (acidosis), metabolic malfunctions, fermentation malfunctions, and/or a microbial imbalance. Other illnesses, for example ketosis or milk fever, or various deficiencies can likewise be treated. This generally includes energy deficiencies, mineral deficiencies (particularly deficiencies of magnesium, phosphorus, calcium, or potassium), vitamin deficiencies, and deficiencies of trace elements.

Advantageous are those substances that are not pharmacologically effective and thus do not mean restriction times for the milk and/or the edible tissue. Nonetheless, the administration of pharmacological substances through the bolus is also possible, for example, with the aid of at least one binding agent as a carrier. This particularly applies to those pharmacological substances affecting appetite.

As will be explained in more detail subsequently, the at least one binding agent may itself have a function necessary for animal health. If protein, sugar, or fat is used for the at least one binding agent, it offers an energetic function so to speak, in addition to the described functions.

As soon as the bolus has reached the forestomach, the structure of the bolus should disintegrate. The components of the bolus should dissolve in the forestomach in this case. The disintegration process can obviously quickly and chronologically follow the act of swallowing. It is also possible for the components of the bolus to dissolve more slowly, particularly at a speed that is determined by the disintegration process of the at least one binding agent. Thus, there is a chronologically continual release of the substances that are necessary for animal health, which are contained in the bolus. The stimulation of an animal's appetite, particularly triggered by raw fiber contained in the bolus, thus does not proceed uncontrollably.

Essentially, the bolus cannot only be used for cattle but any ruminant, for example goats, sheep, and even wild ruminants, for example reindeer and deer. The size of the bolus may vary in this case, particularly vary to the benefit of the type of animal. Typical dimensions for cattle are: diameter 35-40 mm, length 160 mm; typical dimensions for sheep and goats are: diameter 15 mm, length 70 mm.

The bolus preferably has the basic shape of a capsule; this means that it is cylindrical and has ends rounded off toward the exterior. The basic shape here characterizes the essential shape of the bolus as it could be given, for example, as a result of a mold during a treatment process. This means that proportions of the bolus may protrude beyond the basic shape, as is also described in the following.

The basic shape of a capsule essentially provides good gliding properties, particularly due to the rounded-off ends. The basic shape of the capsule, however, is not the only possible basic shape. The bolus may particularly have also a basic shape of an ellipsoid, a cuboid, a cube, a sphere, or a cylinder. This does not exclude any other shaping.

In one advantageous embodiment, a mixture of raw fibers in the at least one binding agent amounts to a proportion of at least 20% of the volume of the bolus. Preferably, the mixture of raw fibers and at least one binding agent has a proportion of at least 30% of the volume (% by volume) of the bolus and especially preferably a proportion of at least 40% by volume. Further alternative lower limits result with a proportion of at least 50% by volume, of at least 60% by volume, of at least 70% by volume, of at least 80% by volume, and of at least 90% by volume. An upper limit that can be combined with all of the mentioned lower limits is 100% by volume.

In a further advantageous embodiment, the bolus substantially consists of a mixture of raw fibers and the at least one binding agent. In particular, this means that other components and/or cavities only comprise a small, particularly no more than a single-digit (preferably less than 5% by volume), percent volume fraction of the bolus. The aforementioned advantageous effects on forestomach health and/or on the health of the ruminant similarly result in an especially pronounced manner. Accordingly, a bolus substantially consisting or completely consisting of the mixture of raw fibers and the at least one binding agent is also possible. The term “substantially” particularly means a proportion of the mixture of at least 95% of the volume (% by volume) of the bolus, or at least 96% by volume, or at least 97% by volume, or at least 98% by volume, wherein an upper limit for the term “substantially” is preferably 99.9% by volume or 99.99% by volume. Completely means a proportion of 100% by volume.

The volume fraction of the raw fibers in the mixture of raw fibers in the at least one binding agent preferably amounts to at least 20% of the volume (% by volume) of the mixture, preferably at least 40% by volume. Further alternative lower limits result with a proportion of at least 50% by volume, of at least 60% by volume, of at least 70% by volume, of at least 80% by volume, and of at least 90% by volume. The volume fraction of raw fibers in the mixture comprising raw fibers and the at least one binding agent may amount to 95% by volume, 96% by volume, 97% by volume, 98% by volume, or 99% by volume of the mixture as an upper limit, wherein any of the aforementioned upper limits can be combined with any of the aforementioned lower limits as desired.

Particularly with the aforementioned dimensions of the bolus, noticeable advantageous effects result on forestomach health and/or on the health of the ruminant, particularly when a sufficient quantity of raw fibers is placed in the forestomach. In a previously mentioned embodiment, at least 20% of the volume of the bolus is made up of the mixture comprising raw fibers and the at least one binding agent. Higher proportions are especially advantageous particularly due to the higher proportion of raw fibers in this context.

In a mature cow, the forestomach may reach a volume of more than 100 L. In order to increase effectiveness, it is possible to administer several boli at brief intervals or one after the other in practical use. Regular administration of boli to increase efficiency is also possible.

In a further advantageous embodiment, which may be used alternatively or additionally to the other embodiments mentioned herein, the raw fibers (only the raw fibers without binding agent) make up a proportion of at least 20% of the volume (% by volume) of the bolus. Preferably, the raw fibers have a proportion of at least 30% by volume of the bolus and especially preferably a proportion of at least 40% by volume. Further alternative lower limits result with a proportion of at least 50% by volume, of at least 60% by volume, of at least 70% by volume, of at least 80% by volume, and of at least 90% by volume. It is also possible to have a bolus consisting substantially of raw fibers such that the binding agent only has at most a single-digit percentage of the volume of the bolus (preferably less than 5% by volume). An upper limit for the volume fraction of raw fiber in the bolus may be 99% by volume, or 98% by volume, or 97% by volume, or 96% by volume, or 95% by volume, wherein any of the aforementioned upper limits can be combined with any of the aforementioned lower limits as desired. The aforementioned advantageous effects on forestomach health and/or on the health of the ruminant similarly result as previously described.

In a further advantageous embodiment, the at least one binding agent has hydrophobic properties. This helps to prevent swelling of the bolus and/or individual components of the bolus, particularly raw fibers, due to moisture. This relates particularly to moisture occurring during storage of the bolus in the storage environment, but also to moisture in the mouth area and throat area of the animal. The at least one binding agent with hydrophobic properties thus serves to ensure that the bolus retains its structure during the act of swallowing and does not disintegrate, i.e. that it has a sufficient degree of structural stability during storage and during the act of swallowing. This is thereby ensured in that there is no water absorption of the bolus or only to a small degree during the act of swallowing due to the hydrophobic properties of the at least one binding agent This is associated with the goal that the decomposition process of the bolus does not start until the forestomach of the animal.

In order to form the hydrophobic properties, processing on the surface of the bolus and/or of the at least one binding agent or of a further substance to be added occurs by means of a plasma in order to achieve the hydrophobic properties.

In a further embodiment, the bolus, as a component, has at least one substance or consists of a substance that is selected from the group consisting of fats, proteins, carbohydrates, salts, synthetic high-molecular-weight substances, and/or minerals, and the compounds thereof. In particular, the binding agent has at least one of these components or consists thereof. At least one of the aforementioned components may be added as a further component if it is not already contained in the binding agent. The at least one aforementioned component may be present in any form within the bolus. Thus, it can be added to the raw fiber, which is used for the bolus, in any form and manner, or may be added to the mixture of raw fiber and the at least one binding agent, or added to any other component of the bolus. The at least one aforementioned component may also be present in a separate unit or a separate area within or on the outer skin of the bolus, for example in a cavity or in at least one separate capsule, which is added to the bolus in its interior or on its outer skin, or in another area within the bolus or outside the bolus. This embodiment has the advantage of promoting the fermentative function of microorganisms, which may be added to the bolus as a component or may already be present in the stomach or forestomach of the animal. The availability of fats, proteins, carbohydrates, and/or the trace elements mentioned subsequently support the goal of quick recovery of the forestomach flora. A fat-containing metabolic preparation for administration in the event of energy deficiencies, i.e. a supplementary feedstuff, is also conceivable. This may be, in turn, for example, Ket 40, which is a brand from ufamed AG.

Salts may be organic or inorganic salts. For the purposes of use as a binding agent, the salts may be melted, solidified, and/or pressed, optionally under the effect of temperature.

Suitable salts, for example, are salts with the calcium, potassium, magnesium, and sodium cations, and the chloride, phosphate, hydrogen sulfate, sulfate, sulfide, carbonate, hydrogen carbonate, nitrite, nitrate, or fluoride anions, wherein these cations and anions may be combined in any manner.

Some salts, for example calcium chloride or potassium chloride, are formed into a structurally very solid and compressed composite during a pressing process within a manufacturing method and are therefore particularly well-suited for use as the at least one binding agent.

It is advantageous if a sponge effect due to swelling can be avoided during production (particularly during a pressing process) of the bolus, particularly with respect to the raw fibers. This is achieved in that the at least one binding agent is composed at least proportionally of one or more of the aforementioned substances provided they are suitable for preventing the aforementioned swelling effect. For example, fats, proteins, and carbohydrates are suitable for this which also have significance as an energy supplier for the animal as it were and can function as a starter for the digestive processes. Explanation: For the functionality of the microorganisms in the forestomach, fats, proteins, and carbohydrates, for example, are especially advantageous in conjunction with raw fibers.

The substances mentioned in the previous paragraph are furthermore suitable for binding and/or bonding and/or affixing the raw fibers. These also include some synthetic high-molecular-weight substances. Such substances are polymers, for example. Substances that are biodegradable in the digestive system of the ruminant as well as substances that are not biodegradable there may be used. Especially preferred are substances that are not biodegradable in the digestive system of the ruminant, i.e. that go through the body of the animal in a chemically inert manner.

Some of the aforementioned substances are furthermore suitable for supplying microorganisms in a forestomach with energy. These are, for example, fats, carbohydrates, proteins, and minerals (for example, calcium, chlorine, potassium, magnesium, sodium, phosphorus, copper, hydrogen carbonates, sulfates, nitrates, fluorides).

The bolus may contain a buffer substance, whereby a pH value can be kept constant in the forestomach of the ruminant. A hydrogen carbonate is an example of this.

In a further embodiment, the raw fibers used in the bolus have plant parts or consist of plant parts. Plant parts represent the natural feed of ruminants and are suitable for activating and supporting the natural digestive processes due to the contained cellulose that is provided in the forestomach. As explained in the sections regarding the next embodiment, hay or straw is particularly suitable for this. In addition to these, essentially other types of processed or unprocessed plant parts, however, are suitable for use in the bolus provided they activate and/or support the natural digestive processes and are animal-friendly.

In a further advantageous embodiment, the plant parts are hay and/or straw. Plant parts consisting of hay and/or straw are especially well-suited for activating and supporting the natural digestive processes because, in addition to their cellulose content, they stimulate the natural digestive processes on the walls of a forestomach due to mechanical stimuli.

The hay and/or straw may be an older or newer hay and/or older or newer straw as relates to the harvest and/or processing and/or to the age of the harvested/processed plants. If the hay and/or straw is older, the digestibility is generally less but the structural integrity is higher. If the hay and/or straw is newer, the digestibility is generally higher but the structural integrity is less. Proportionally older or newer hay and/or proportionally older or newer straw may be used as the raw fiber in the bolus depending on the intended purpose.

In a further advantageous embodiment, the length of the raw fibers is from 3 cm to 8 cm, preferably 5 cm to 8 cm. This length may be adjusted, for example, by the chop. It has been shown that the stimulation described in the previous paragraph is used to a great degree with this length. The stipulation of 3 cm to 8 cm, preferably 5 cm to 8 cm, is not a strictly defined dimension but only basic information. It is also possible to have shorter or longer components in the raw fiber despite the indicated chop length.

Further possible components of the bolus are mentioned in the following. Further effects can be achieved and/or application synergies can be reached with these components. For example, needle injections can be avoided or reduced when administering vitamins or pharmacologically effective substances as mentioned in the following. Other advantages are listed at the particular point.

Convalescence synergies with the bolus can be achieved in the following manner with the adding of one or more of the additional, subsequently listed components: Primary or secondary diseases of the forestomach lead directly (failure of the microbiological flora) or indirectly, due to metabolic malfunctions, to a reduction in feed consumption and performance of the animal. In addition, excessive anti-inflammatory products will generally have a damping effect on feed consumption in a similar manner. If the uppermost goal, the improvement of feed consumption, is then intended, energetic deficiencies, etc. can be compensated for, depending on the incidence of disease, by adding certain substances such as, for example, minerals, particularly calcium, phosphorus, potassium, or vitamins and/or excessive anti-inflammatory products can be neutralized.

Thus, in special embodiments, it is possible with the invention to compensate for the negative primary and secondary effects of derailed forestomach health or to counteract it. This is also called recovery synergy (re-convalescence synergy). On the one hand, a raw fiber deficiency can be compensated for and, furthermore, secondary metabolic incidences of illness can be treated by one or more of the aforementioned further components in order to counteract particularly the cause of the raw fiber deficiency. A synergy can hereby be achieved in feed consumption, stimulation of appetite, and possibly re-convalescence.

Trace Element:

In a further advantageous embodiment, the bolus contains at least one trace element, particularly from the group consisting of cobalt, iron, fluorine, iodine, copper, manganese, molybdenum, selenium, silicon, vanadium, zinc, chromium, and the compounds thereof. Such a trace element may be added as a further component if the binding agent does not already contain such. The at least one trace element may be present in any form within the bolus. Thus, it can be added to the raw fiber, which is used for the bolus, in any form and manner, or may be added to the mixture of raw fiber and the at least one binding agent, or added to any other component of the bolus. The at least one trace element may also be present in a separate unit or in a separate area within or on the outer skin of the bolus, for example in a cavity within the structure of the bolus or in at least one separate capsule, which is added to the bolus in its interior or on its outer skin, or in another area within the bolus or outside the bolus.

In particular, the at least one trace element may be mixed with the at least one binding agent or/and the mixture of raw fiber and at least one binding agent or/and with at least one other component of the bolus. On the one hand, this is significant with respect to a production process of the bolus, because the production process is especially simple due to a mere adding and mixing with the at least one trace element. On the other hand, there is the advantage that the at least one trace element with the at least one binding agent can be released in the forestomach in a chronologically continual form and manner, according to the disintegration process of the at least one binding agent. The chronologically continual form and manner of the release may promote animal health.

The trace element itself may likewise be part of the at least one binding agent or may be contained in a component of the at least one binding agent. The advantages described previously result similarly.

Vitamin

In a further advantageous embodiment, the bolus contains at least one vitamin, particularly from the group consisting of vitamin A, vitamin D, vitamin E, vitamin K1, vitamin K2, vitamin K3, vitamin K4, vitamin C, vitamin B1, vitamin B2, vitamin B3, vitamin B5, vitamin B6, vitamin B7, vitamin B9, vitamin B12. Such a vitamin may be added as a further component if the binding agent does not already contain such. The at least one trace element may be present in any form within the bolus. Thus, it can be added to the raw fiber, which is used for the bolus, in any form and manner, or may be added to the mixture of raw fiber and the at least one binding agent, or added to any other component of the bolus. The at least one vitamin may also be present in a separate unit or in a separate area within or on the outer skin of the bolus, for example in a cavity or in at least one separate capsule, which is added to the bolus in its interior or on its outer skin, or in another area within the bolus or outside the bolus.

In particular, the at least one vitamin may be mixed with the at least one binding agent or/and the mixture of raw fiber and at least one binding agent or/and with at least one other component of the bolus. On the one hand, this is significant with respect to a production process of the bolus, because the production process is especially simple due to a mere adding and mixing with the at least one vitamin. On the other hand, there is the advantage that the at least one vitamin with the at least one binding agent can be released in the forestomach in a chronologically continual form and manner, according to the disintegration process of the at least one binding agent. The chronologically continual form and manner of the release may promote animal health.

The vitamin itself may likewise be part of the at least one binding agent or may be contained in a component of the at least one binding agent. The advantages described previously result similarly.

Agent Aiding Health and/or Digestion

In a further advantageous embodiment, the bolus contains at least one agent aiding health and/or digestion, particularly from the group consisting of magnesium and magnesium compounds, phosphorus and phosphorus compounds, calcium and calcium compounds, potassium and potassium compounds, hydrogen carbonates, herbal extracts, essential oils, and pharmacologically effective substances. Such an agent aiding health and/or digestion may be added as a further component if the binding agent does not already contain such.

The at least one agent aiding health and/or digestion may be present in any form within the bolus. Thus, it can be added to the raw fiber, which is used for the bolus, in any form and manner, or may be added to the mixture of raw fiber and the at least one binding agent, or added to any other component of the bolus. The at least one agent aiding health and/or digestion may also be present in a separate unit or in a separate area within or on the outer skin of the bolus, for example in a cavity or in at least one separate capsule, which is added to the bolus in its interior or on its outer skin, or in another area within the bolus or outside the bolus.

In particular, the at least one agent aiding health and/or digestion may be mixed with the at least one binding agent or/and the mixture of raw fiber and at least one binding agent or/and with at least one other component of the bolus. On the one hand, this is significant with respect to a production process of the bolus, because the production process is especially simple due to a mere adding and mixing with the at least one agent aiding health and/or digestion. On the other hand, there is the advantage that the at least one agent aiding health and/or digestion with the at least one binding agent can be released in the forestomach in a chronologically continual form and manner, according to the disintegration process of the at least one binding agent. The chronologically continual form and manner of the release may promote animal health.

The agent aiding health and/or digestion itself may likewise be part of the at least one binding agent or may be contained in a component of the at least one binding agent. The advantages described previously result similarly.

Yeast, Microbiological Starter Cultures

In a further advantageous embodiment, the bolus contains yeast and/or microbiological starter cultures as a component.

A microbiological starter culture has the function and is used to initiate the digestive processes in a forestomach and may further serve to stabilize and aid the microbiological integrity in the forestomach. An example of a microbiological starter culture is a so-called rumen starter.

Yeast, which is listed separately from the microbiological starter culture, may have a function similar to that of the microbiological starter culture and, in doing so, eliminate excess, easily soluble carbohydrates.

At least one of the aforementioned components may be added as a further component if it is not already contained in the binding agent. The at least one aforementioned component may be present in any form within the bolus. Thus, it can be added to the raw fiber, which is used for the bolus, in any form and manner, or may be added to the mixture of raw fiber and the at least one binding agent, or added to any other component of the bolus. The at least one aforementioned component may also be present in a separate unit or a separate area within or on the outer skin of the bolus, for example in a cavity or in at least one separate capsule, which is added to the bolus in its interior or on its outer skin, or in another area within the bolus or outside the bolus.

A microbiological starter culture may consist particularly of microorganisms. The microorganisms may have, in particular, one or more of the following properties: They may be selected based on specific properties. The microorganisms may be capable of propagation. The microorganisms may have a supporting effect with fermentative processes in the forestomach.

The microbiological starter culture may have, for example, bacteria, for example and without limitation, lactic acid bacteria, and/or fungi (particularly different than the yeast).

In particular, a yeast fungus or a yeast fungus preparation may furthermore contain carbohydrates, fat, and protein, i.e. be used as a varied energy supplier. Furthermore, an additional binding effect is possible due to the protein contained.

A sufficient degree of advantageous metabolic processes in the forestomach of a ruminant depends decisively on sufficient supply of raw fibers. Such an advantageous metabolic process is, in particular, the fermentation of carbohydrates, particularly of cellulose, by microorganisms. Such advantageous metabolic processes are enhanced and/or started by microbiological starter cultures. The metabolization of the raw fibers can be catalyzed by yeast and/or microbiological starter culture. Starter cultures or yeast in this case primarily serve as catalysts for the physiological microbiological fermentation.

A sick animal, for example a cow, typically stops eating, whereby the supply of dietary fibers in the forestomach is stopped. This results in a raw fiber deficiency, which leads to fermentation problems in the forestomach, which becomes noticeable, for example, in acidification (acidosis). The bolus according to the invention is used to supply necessary raw fibers. If the condition in the forestomach is already compromised however, a certain amount of time is required until the supplied raw fiber is metabolized. By adding yeast and/or microbiological starter cultures to the bolus, they are introduced into the forestomach with the bolus and can improve the microbiological flora there and stimulate the metabolic processes in the forestomach such that the compromised condition can be more quickly improved and health can be more quickly achieved.

Thus, the simultaneous supply of structurally effective raw fiber and microbiological starter cultures and/or yeasts provides an especially advantageous combination of various active mechanisms. There is a synergy in this regard in that the microbiological starter culture and/or yeast works together with the raw fiber in order to metabolize the latter.

In addition, animal-friendly structural effectiveness of the raw fiber is advantageous for implementing the advantageous metabolic processes. Structural effectiveness particularly means that the particle length and hardness have an advantageous mechanical effect on the forestomach and stimulates it to contract. The ruminant is hereby stimulated to ruminate.

In particular, the at least one listed component, which is selected from the group consisting of yeast and/or microbiological starter cultures, may be mixed with the at least one binding agent or/and the mixture of raw fiber and at least one binding agent or/and with at least one other component of the bolus. On the one hand, this is significant with respect to a production process of the bolus, because the production process is especially simple due to a mere adding and mixing with the at least one substance mentioned. On the other hand, there is the advantage that the at least one substance mentioned can be released in the forestomach in a chronologically continual form and manner, according to the disintegration process of the at least one binding agent. The chronologically continual form and manner of the release may promote animal health.

The listed substance itself may likewise be part of the at least one binding agent or may be contained in a component of the at least one binding agent. The advantages described previously result similarly.

The adding of at least one previously mentioned trace element and/or a previously mentioned vitamin and/or a previously mentioned agent aiding in health and/or digestion may take place in isolation or, in an especially advantageous embodiment, in addition to yeast and/or microbiological starter cultures. As previously mentioned, yeasts and microbiological starter cultures serve advantageously to accelerate the metabolism of supplied raw fiber in the forestomach.

A microbiological imbalance in the forestomach, which may particularly result in acidification of the forestomach, is a result of an illness or a prerequisite for the development of further illnesses of the ruminant in many cases (for example, inflammation of the lungs and/or other body parts). A restoring of a healthy forestomach function promotes recovery from a previously existing illness or is a prerequisite for preventing or recovering from further illnesses, which may occur only after the compromise of the forestomach function.

In order to treat an illness existing before the compromise of forestomach function, or an illness subsequently resulting, a pharmacologically effective substance, for example a medicine, particularly may be added to the bolus as a further component as an agent aiding the health. For prevention, an agent that aids health in some other manner, for example an herbal extract, may be added.

A bolus, which contains at least one such further substance in addition to yeast and/or microbiological starter culture, thus takes on at least one dual function in the mechanism of action; there is an application synergy with respect to restoring the forestomach function and recovery from or prevention of an illness.

Furthermore, the adding of several active substances in one use of the bolus promotes animal welfare: Ruminants then need treatment, for example, by means of injection, that is potentially unpleasant or painful to them less often; if a combination of active substances is present in the bolus, several active substances can thus be administered simultaneously.

In a further advantageous embodiment, raw fiber protrudes proportionally from the surface of the bolus, i.e. proportionally over the basic shape of the bolus. This then results in an irregular, uneven outer contour of the bolus. The decomposition of the bolus in the forestomach is hereby facilitated, because the application surface for dissolving liquids is increased.

Furthermore, a production method of the bolus is proposed, wherein raw fiber and the at least one binding agent are mixed and molded into a bolus.

A bolus according to any of the previously described embodiments can be produced with the method. Features previously disclosed according to the method by means of a bolus may be a component of a method according to the invention. Previously mentioned features can be used in the method or thereby created.

The mixing and/or the molding may take place under the effects pressure and/or temperature in a further embodiment of the production method.

Further components or parts of the bolus can be added to the mixture comprising raw fiber and the at least one binding agent at any time in the production method.

In this case, a mixing of the components or parts may take place under mechanical action, for example with the aid of a motor-driven or muscle-driven or by means of a hydraulically or pneumatically functioning mixing and/or pressing and/or molding unit. The mixing process may take place under the effect of a temperature that is different from the ambient temperature, particularly that is higher than the ambient temperature.

During the molding process, the mixture of raw fiber and the at least one binding agent and/or further components or parts under mechanical action takes place, for example, with the aid of a motor-driven or muscle-driven or by means of a hydraulically or pneumatically functioning mixing and/or pressing and/or molding unit. The molding process may take place under the effect of a temperature that is different from the ambient temperature, particularly that is higher than the ambient temperature.

Over the course of the production method, at least one mixing process of the mixture may additionally be implemented with further components or parts. At least one separate mixing process of the further components may also be implemented before the mixing process with the mixture. The resulting mixture of further components may then be added to the mixture in any form and at any time.

An exemplary embodiment of the invention will now be described with reference to the enclosed drawing. The individual figures in the drawing show the following:

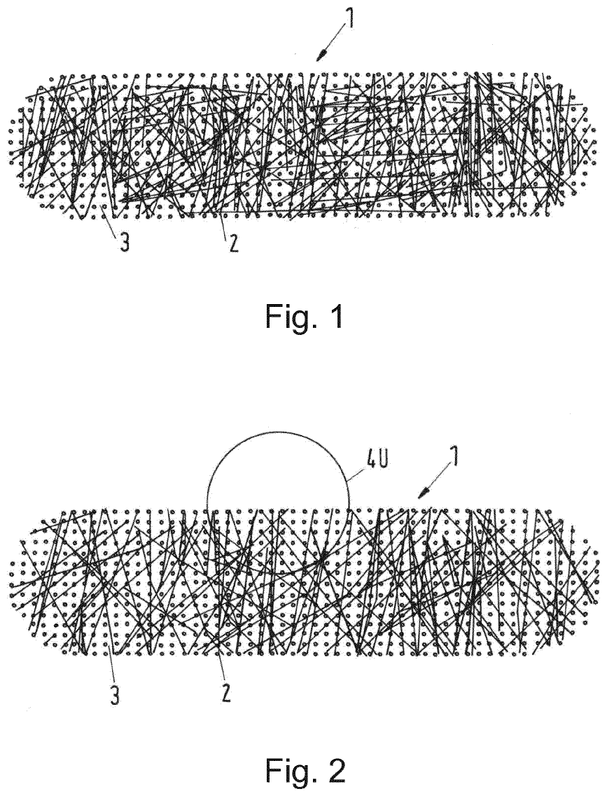

FIG. 1 a capsule-shaped bolus according to the invention, which has a mixture of raw fiber and at least one binding agent, viewed from the outside in the longitudinal direction.

FIG. 2 a longitudinal section through the bolus shown in FIG. 1, wherein an edge area is indicated by a circular outline;

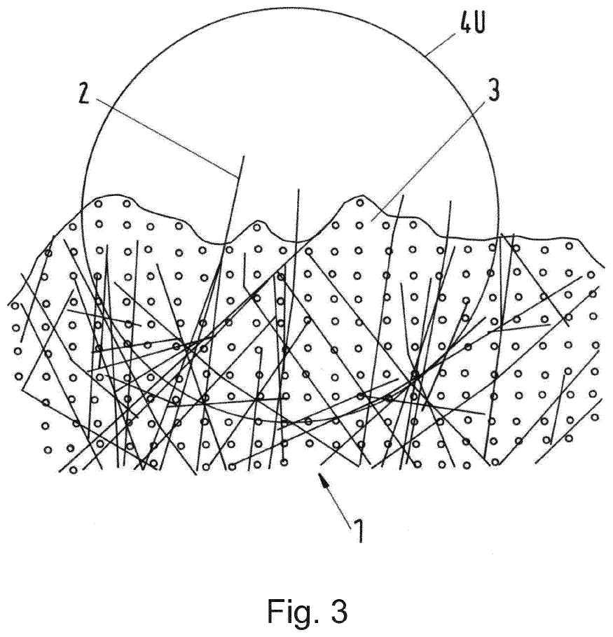

FIG. 3 the edge area of the longitudinal section through the bolus shown in FIG. 2, wherein surrounding sub-sections are shown outside of the marking;

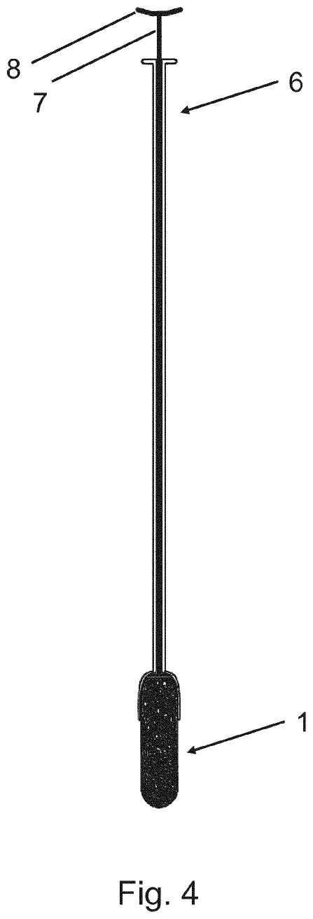

FIG. 4 the capsule-shaped bolus (1) according to the invention, which is shown in FIGS. 1-3, in a customary administration aid before the administration process;

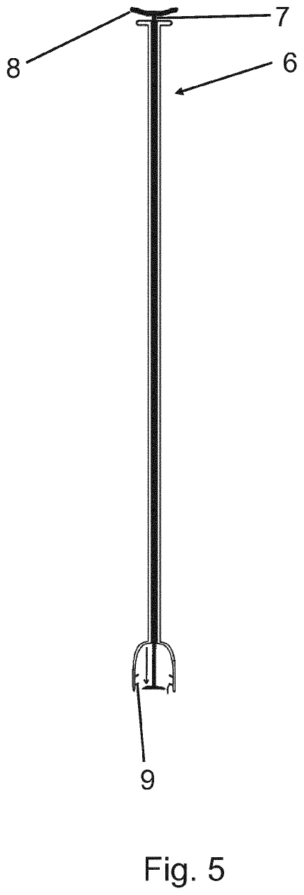

FIG. 5 the customary administration aid, which is already shown in FIG. 4, after the administration process.

The bolus 1, which has the three-dimensional shape of a capsule, i.e. is circular in cross-section and rounded off like a hemisphere at the ends, consists of a mixture of raw fiber 2 and at least one binding agent 3. This is shown schematically in FIG. 1, wherein the raw fiber 2 is shown in the form of dark lines and the binding agent 3 is shown as a dotted, continuous region. The raw fiber 2 is densely packed due to the described production method. The binding agent 3 is located between the individual components of the raw fiber 2 and exerts its function of affixing the components of the raw fiber 2 and connecting them to one another. In reality, the density of the raw fiber 2 is preferably higher than what is shown in the figures for the sake of clarity.

FIG. 2 shows a longitudinal section through the bolus 1. The regions containing the binding agent 3 are shown between the dark lines, which represent the raw fiber. Furthermore, an edge area of the bolus 1 is marked by a circular outline 4U.

FIG. 3 shows the edge area of the bolus 1 enlarged, which is shown by the circular outline 4U in FIG. 2. It can clearly be seen here that the outer contour of the bolus 1 does not extend completely and not exactly straight everywhere even if this is shown and/or looks this way in FIG. 2 for the sake of simplicity. It can be seen that individual components of the raw fiber 2 protrude from the surface, which is formed by the contiguous binding agent 3 and is shown by a thin, dark contour line. The outer contour of the bolus 1, formed by the individual components of the raw fiber and the binding agent, is irregular and not completely smooth. Such type of outer contour is in the nature of the components used of the bolus 1 and also in the nature of the production method.

FIG. 4 shows the bolus 1 used in a customary administration aid 6. If the pressure pin 7 at the top of the administration aid 6 exerts pressure onto the handpiece 8, the bolus 1 is repelled. The administration aid 6 in this case is located in the mouth and throat area and in the esophagus of the animal.

FIG. 5 shows the administration aid 6 after repelling of the bolus 1. The handpiece 8 has been actuated. The bolus 1, which was retained in the administration aid 6 by clamp-like hooks 9, is repelled.

### LIST OF REFERENCE NUMERALS

1 Bolus
2 Raw fiber
3 Binding agent
4U Circular outline
6 Administration aid
7 Pressure pin
8 Handpiece
9 Clamp-like hooks

1. A bolus for administration to a forestomach of a ruminant, wherein the bolus comprises a mixture of raw fibers and at least one binding agent.

2. The bolus according to claim 1, wherein the mixture of raw fibers and the at least one binding agent amounts to a proportion of at least 20% of the volume of the bolus.

3. The bolus according to claim 1, wherein raw fibers amount to a proportion of at least 20% of the volume of the bolus.

4. The bolus according to claim 1, wherein the bolus substantially consists of a mixture of raw fibers and the at least one binding agent.

5. The bolus according to claim 1, wherein the at least one binding agent has hydrophobic properties.

6. The bolus according to claim 1, wherein the at least one binding agent comprises fats, proteins, carbohydrates, salts, synthetic high-molecular-weight substances, and/or minerals, and compounds thereof.

7. The bolus according to claim 1, wherein the raw fiber comprises plant parts or consists of plant parts.

8. The bolus according to claim 7, wherein the plant parts are hay and/or straw.

9. The bolus according to claim 1, wherein the length of the raw fibers is 3 cm to 8 cm.

10. The bolus according to claim 1, wherein the bolus comprises yeast and/or microbiological starter cultures.

11. The bolus according to claim 1, wherein the bolus comprises at least one trace element.

12. The bolus according to claim 1, wherein the bolus comprises a vitamin.

13. The bolus according to claim 1, wherein the bolus comprises at least one agent aiding health and/or digestion.

14. The bolus according to claim 10, wherein the yeast and/or microbiological starter cultures is mixed with the at least one binding agent and/or the mixture of raw fibers and the at least one binding agent and/or with at least one other, further component of the bolus.

15. The bolus according to claim 1, wherein raw fiber protrudes proportionally from the surface of the bolus.

16. A method for producing a bolus according to claim 1, comprising mixing raw fiber and the at least one binding agent together and molding the mixture into a bolus.

17. The method according to claim 16, wherein the mixing and/or the molding take(s) place under the effect of pressure and/or temperature.

18. The bolus according to claim 1, wherein the bolus has the shape of a capsule.

19. The bolus according to claim 1, wherein the bolus has the shape of an ellipsoid, a cuboid, a cube, a sphere or a cylinder.

20. The bolus according to claim 1, wherein the bolus is in a shape so that the bolus can be entirely swallowed by the ruminant.

21. A method to stabilize and/or stimulate feed consumption in a ruminant comprising administering the bolus of claim 1 to the forestomach of the ruminant.

22. The method according to claim 21, wherein the raw fibers stimulate the forestomach by mechanical stimuli on the walls of the forestomach.
